(19) United States
(12) Patent Application Publication
(10) Pub. No.: US 2019/0209190 A1
(43) Pub. Date: Jul. 11, 2019

(54) THROMBUS RETRIEVER

(71) Applicant: MEDINOL LTD., Tel Aviv (IL)

(72) Inventor: Jacob RICHTER, Arsuf (IL)

(21) Appl. No.: 16/333,174

(22) PCT Filed: Sep. 5, 2017
(86) PCT No.: PCT/IB2017/001225

§ 371 Date: Mar. 13, 2019

Related U.S. Application Data
(60) Provisional application No. 62/395,217, filed on Sep. 15, 2016.

(51) Int. Cl. A61B 17/221; A61B 17/3207

(52) U.S. Cl. CPC A61B 17/221; A61B 2017/2215; A61B 2017/2212; A61B 17/32075

(57) ABSTRACT

The invention provides a thrombus retriever and associated methods for use. The thrombus retriever has a retriever body that is attached to a centrally-disposed deployment member capable of moving the retriever body in the proximal direction (towards the operator) in order to retrieve the target thrombus. The retriever body is attached to the deployment member at an attachment point distal to the most distal end of the retriever body. In use, pulling the deployment member in the proximal direction effectively “pushes” the retriever body from behind during thrombus collection. The thrombus retriever also may comprise a net at its distal end to collect the retrieved thrombus and prevent embolization.

### CROSS-REFERENCE TO RELATED APPLICATIONS

This application claims priority to the U.S. provisional patent application Ser. No. 62/395,217, filed Sep. 15, 2016. Priority to the provisional patent application is expressly claimed, and the disclosure of the provisional application is hereby incorporated herein by reference in their entireties and for all purposes.

### FIELD OF THE INVENTION

This invention relates generally to devices deployed from endovascular catheters for removing thrombi, and methods for using the same.

### BACKGROUND OF THE INVENTION

Thrombi, also known as “blood clots,” can form in both arterial and venous vessels. Thrombi, when formed in or embolized into such vessels can cause adverse events by blocking the blood flow downstream in the blocked vessel.

Thrombi may be treated/removed by thrombolytic therapy and/or mechanical thrombectomy. Thrombolysis dissolves a thrombus using pharmacological agents such as tissue plasminogen activator drug (tPA). Catheter-delivered thrombectomy devices generally fall into two categories and combinations thereof. Aspiration catheters function by suctioning the thrombus out of the blood vessel. Mechanical devices are designed to contact and dislodge the thrombus, and guide it into the catheter sheath or an aspiration catheter for removal from the body. These devices include balloon catheters that are designed to be pushed through the thrombus, inflated, and withdrawn to pull the thrombus into the removal catheter. Other devices are designed to entangle or otherwise capture the thrombus within the blood vessel using a corkscrew or spring/coil device, and then move the thrombus into the catheter sheath for removal.

The structure of a thrombus is complex and may include platelets, blood cells, and fibrin. Thrombi tend to have a hard and dense perimeter lying against the vessel wall, and a liquid or gelatinous core towards the center of the vessel lumen. Existing thrombectomy devices are inefficient or incapable of removing the entirety of a thrombus. For example, aspiration catheters may remove the softer, central portions of the thrombus, thereby partially restoring vessel patency, but may not provide enough suction to dislodge the dense perimeter portions of the thrombus. The mechanical capture devices that entangle or snare a thrombus may be effective at removing relatively dense thrombi but are incapable of gaining purchase on softer thrombi while increasing the risk of embolism. Further, as these mechanical devices are moved proximally along a vessel in order to collect a thrombus, the denser perimeter portions of the thrombus may force the device to decrease in diameter. This may cause the mechanical device to not only fail to collect the softer thrombus, but also a portion of the harder thrombus adjacent to the vessel wall. is the relative inefficiency of collection may necessitation repeated retriever pulls and even then may result in incomplete thrombus removal. Thus, there is a need for a retriever that is capable of efficiently removing the entirety, or nearly the entirety, of a thrombus in a single pull, while minimizing the risk of embolism.

### SUMMARY OF THE INVENTION

The invention provides a thrombus retriever and associated methods of use. The thrombus retriever generally may consist of a retriever body, a mesh covering at least the distal end of the retriever body, a centrally-disposed deployment member, and a centrally-disposed collecting member; the latter two elements being under independent operator control. The retriever body is attached through leading arms to the deployment member at a point that is more distal than the distal-most edge of the retriever body, thereby pulling the device from its distal edge thus directing it towards the vessel wall resulting in more complete collection of thrombus. The thrombus retriever is designed and adapted to be housed in, and deployed from an intravascular catheter. Accordingly, the thrombus retriever and, particularly the retriever body, is capable of adopting a crimped conformation (e.g., when loaded into a catheter) and a deployed conformation (e.g., when deployed within a blood vessel for thrombus collection). The crimped conformation, therefore, has a smaller diameter or width than the deployed conformation. Various embodiments and features of the thrombus retriever are described below.

In one aspect, the invention provides a thrombus retriever having:
a substantially cylindrical retriever body open at a proximal end, open at a distal end, having a longitudinal axis, and capable of adopting a first crimped conformation having a first diameter and a deployed conformation having a second diameter, and wherein the first diameter is smaller than the second diameter,
a mesh covering at least the distal end of the retriever body,
a deployment member extending through the retriever body along an axis substantially parallel to the longitudinal axis, and
a collecting member attached to the retriever body and adapted to cause the retriever body to adopt a second crimped conformation, a collected conformation whereby the retriever body with its mesh and the already collected thrombus are collected into the catheter. (e.g., a crimped or partially-crimped conformation) from the deployed conformation, wherein the collected conformation has a third diameter that is smaller than the second diameter,
wherein the retriever body is attached to the deployment member at an attachment point distal to the distal end of the retriever body thus being pushed through the thrombus rather than being pulled; and
wherein the deployment member and the collecting member are capable of independent movement in the longitudinal direction.

In another embodiment, the retriever body also has two or more leading arms extending from the proximal end of the retriever body and that are connected to the collecting member. Optionally, each of the leading arms is attached to the collecting member by one or more flexible wires.

In some embodiments, the third diameter is greater than, or substantially the same as the first diameter.

In a second aspect, the invention provides a thrombus retriever having:
a substantially cylindrical retriever body open at a proximal end, open at a distal end, having a longitudinal axis, and capable of adopting a crimped conformation having a first diameter and a deployed conformation having a second diameter, and wherein the first diameter is smaller than the second diameter, wherein the retriever body further comprises leading arms extending from the proximal end,
a mesh covering at least the distal end of the retriever body, and
an actuator extending through the retriever body along an axis substantially parallel to the longitudinal axis,
wherein the retriever body is attached to the actuator at a distal attachment point distal to the distal end of the retriever body to guide the retriever through the thrombus without decreasing its diameter by pushing it from behind rather than pulling it through the thrombus, and
wherein the leading arms are attached to the actuator by a plurality of flexible wires at a proximal attachment point proximal to the proximal end of the retriever body to allow the collection of thrombus into the catheter.

In one embodiment of any of the thrombus retrievers described above, the retriever body comprises one or more first circumferential bands having loops forming a generally sinusoidal or zig-zag pattern around the circumference of the retriever body.

In another embodiment of any of the thrombus retrievers described above, the retriever body comprises one or more second circumferential bands having loops forming a generally sinusoidal or zig-zag pattern around the circumference of the retriever body. The first circumferential bands and the second circumferential bands may have the same or different generally sinusoidal patterns. Optionally, the loops of the one or more first circumferential bands are out of phase with the loops of the first circumferential bands. Optionally, the arms of the sinusoidal ring between loops is sigmoidal rather than straight in order to allow a smaller and denser crimped diameter.

In a third aspect, the invention provides a thrombus retriever having:
a substantially circular retriever body open at a proximal end, open at a distal end, having a longitudinal axis, and capable of adopting a crimped conformation and a deployed conformation, wherein the retriever body is a hoop with a round or substantially round (e.g., oval) cross-section made of round wire or a ribbon,
a mesh covering at least the distal end of the retriever body,
a deployment member extending through the retriever body along an axis substantially parallel to the longitudinal axis, and
a collecting member attached to the retriever body and adapted to cause the retriever body to resume the crimped conformation from the deployed conformation,
wherein the retriever body is attached to the deployment member at an attachment point distal to the distal end of the retriever body, allowing it to be pushed through the thrombus rather than being pulled; and
wherein the deployment member and the collecting member are capable of independent translocation in the longitudinal direction.

In one embodiment, the retriever hoop plane is canted at an angle of 0°-45° (e.g., about 0°, 5°, 10°, 15°, 20°, 25°, 30°, 35°, 40°, or 45°) relative to the deployment member or the longitudinal axis or lumen wall of the blood vessel in which the retriever is deployed.

In another embodiment, the retriever hoop is a ribbon having a proximal edge and a distal edge, wherein the distal edge is medially-disposed relative to the proximal edge such that the ribbon forms a canting angle of 135°-180° (e.g., about 135°, 140°, 145°, 150°, 155°, 160°, 165°, 170°, 175°, or 180°) to allow it to plow into the denser thrombus close to the vessel wall.

In some embodiments of the thrombus retrievers described herein, the retriever body is self-expanding. In some embodiments, the crimped conformation of the retriever body has a diameter of 1-5 French (e.g., 2-4 French). In some embodiments, the deployed conformation of the retriever body has a diameter of 3-12 French (e.g., about 3, 6, 9, or 12 French).

In other embodiments of the inventive thrombus retrievers, the retriever body is attached to the deployment member by two or more (e.g., three, four, five, six or more) distal arms. The distal arms are attached to the retriever body on their proximal ends and to the deployment member on their distal ends, thereby extending from the distal end of the retriever body and thereby serving to push the retriever body from behind when it is pulled through the thrombus. The distal arms may be fabricated as separate pieces and attached to the retriever body and deployment member (e.g., by welding). Alternatively, the distal arms may be fabricated as elements contiguous with either the retriever body or the deployment member. Optionally, the distal arms, by pushing the retriever body from behind, exert an outwardly-directed force on the retriever body, pushing it towards the vessel wall. Such outward force provides a bias for the retriever body toward the vessel wall (i.e., toward a larger-diameter conformation) to secure more complete retrieval of dense thrombus at the vessel wall.

In other embodiments of the inventive thrombus retrievers, the deployment member is a rigid or semi-rigid wire.

In other embodiments of the inventive thrombus retrievers, the collecting member is a tube comprising a lumen. Optionally, the deployment member is a rigid or semi-rigid wire that passes through the lumen of the tubular collecting member.

In some embodiments of the inventive thrombus retrievers, the net or mesh is a woven net, a perforated membrane or a non-woven mesh of polymer fibers. bursiform.

In other embodiments, any portion of thrombus retriever is made from a shape-memory material including, for example, shape-memory metals. In some embodiments, the shape-memory metal is nickel titanium (“NiTi” or nitinol).

In another aspect, the invention provides a thrombus retrievers described herein, in a crimped conformation, housed within the lumen of an intravascular catheter. In some embodiments, the catheter is a microcatheter (e.g., having a lumen diameter of 2 French or 3 French).

In another aspect, the invention provides a method for retrieving or removing a thrombus from a blood vessel of a subject (e.g., a human subject). A thrombus retriever described herein is provided, wherein the retriever body is in a crimped conformation and housed within the lumen of an intravascular catheter. The deployment member and the collecting member are independently controlled by the operator (e.g., interventional cardiologist, radiologist or neuro-radiologist). The catheter is pushed in the distal direction through the target thrombus until the catheter lumen opening is positioned across the distal side of the thrombus.

The thrombus retriever then is deployed. In one embodiment, the thrombus retriever is deployed by withdrawing the catheter sheath in the proximal direction while maintaining the deployment member in a fixed position, thereby maintaining the thrombus retriever on the distal side of the target thrombus. In another embodiment, the thrombus retriever is deployed by first displacing it from the catheter by pushing the deployment member in the distal direction while maintaining the catheter in a substantially fixed position relative to the blood vessel. After deployment, the catheter sheath is withdrawn in the proximal direction while maintaining the deployment member in a substantially fixed position. After retriever deployment, the catheter sheath is maintained on the proximal side of the target thrombus, optionally within less than 10 mm, less than 20 mm, or less than 30 mm of the target thrombus. Optionally, the retriever body is self-expanding and expands simultaneously upon withdrawal of the catheter sheath. Alternatively, the retriever body can be expanded by a balloon. After expansion, the balloon can be deflated and withdrawn through the lumen of the collecting member.

The operator then pulls the deployment member in the proximal direction until the retriever body traverses the target thrombus and retrieve the thrombus into the net. Preferably, the catheter sheath remains stationary.

In one embodiment, the operator then pulls the collecting member in the proximal direction sufficient to bring the retriever body into a crimped or partially crimped conformation such that the retriever body is conformed to be collected into the catheter lumen. The deployment member and the collecting member are then simultaneously pulled in the proximal direction such that the retriever body is maintained in the crimped or partially crimped conformation and also is translocated to a position within the catheter lumen, thereby restoring the retriever body to a crimped conformation and collecting the thrombus into the catheter.

In another embodiment, the collecting member and the deployment member are pulled simultaneously in the proximal direction with the collecting member being pulled to a greater extend (i.e., being proximally-translocated relative to the deployment member) such that the retriever body is simultaneously brought into a crimped or partially crimped conformation and translocated in a proximal direction.

Following collection of the retriever body and collected thrombus into the catheter lumen, the operator withdraws the catheter from the subject.

“Proximal” is a relative term that refers to the direction or side towards the operator and the entry point of the catheter into the vessel. For example, an operator withdrawing a catheter from a patient is translating the catheter in the proximal direction. Likewise, the side or face of a thrombus first encountered by a catheter during insertion of the catheter across the thrombus, is the proximal side.

“Distal” is a relative term that refers to the direction or side away from the operator and the entry point. For example, an operator inserting a catheter into a patient is translating the catheter in a distal direction.

### BRIEF DESCRIPTION OF THE DRAWINGS

FIG. 1 is a cross-sectional schematic view of a thrombus retriever deployed on the distal side of a target thrombus contained within a blood vessel and being pulled at the direction of the arrow to collect the thrombus.

FIG. 2A is a perspective view of one embodiment of a thrombus retriever body in a deployed conformation attached to a deployment member (central wire) and a deployment member (inner tube).

FIG. 2B is a perspective view of the thrombus retriever illustrated in FIG. 2A having the retriever body covered by a mesh.

FIG. 2C is a schematic of circumferential bands, in expanded and crimped conformations, useful for constructing retriever bodies in accordance with the principles of this invention.

FIG. 3 is a perspective view of one embodiment of a thrombus retriever in a crimped conformation housed within an intravascular catheter.

FIG. 4 is a cross-sectional schematic of the thrombus retriever illustrated in FIG. 2 deployed on the distal side of a target thrombus contained within a blood vessel and being pulled in the direction of the arrow to collect the thrombus.

FIG. 5 is a perspective view of a thrombus retriever in a partially-collected conformation with wires connected to the leading arms entering the catheter lumen.

FIG. 6 is a perspective view of a thrombus retriever in a further partially-collected conformation in which the leading arms of the retriever body are partially entering the lumen of an intravascular catheter.

FIG. 7 is a perspective view of another embodiment of a thrombus retriever in a crimped conformation housed within an intravascular catheter.

FIG. 8A is a schematic plan view of a thrombus retriever in a deployed conformation in which the retriever body is a ring.

FIG. 8B is a schematic plan view of the thrombus retriever illustrated in FIG. 8A having a distally-attached bursiform net.

FIG. 9 is a cross-sectional schematic illustrating the cross-section of a ribbon made ring-retriever body within a blood vessel lumen.

FIG. 10A is a cross-sectional schematic illustrating the cant of a retriever body relative to the blood vessel wall in which the retriever body is disposed at an angle of less than 180°.

FIG. 10B is a cross-sectional schematic illustrating a retriever body disposed substantially parallel (i.e., cant angle=about 180°) to the blood vessel wall.

FIG. 11 is a top view illustrating the relationship of the retriever body to the distal arms and deployment member.

FIG. 12 is a schematic perspective view of a ring thrombus retriever in a deployed conformation.

FIG. 13A is a perspective view of a ring thrombus retriever in a crimped conformation housed within an intravascular catheter.

FIG. 13B is a perspective view of a thrombus retriever in a partially-deployed or partially collected conformation in which the proximal end of the retriever body is partially disposed within the lumen of an intravascular catheter and the distal end of the retriever body is defected in the proximal direction toward the centrally-disposed deployment member.

FIG. 13C is a perspective view of a thrombus retriever in a partially-deployed or partially collected conformation in which the proximal end of the retriever body (the ring) is further disposed within the lumen of an intravascular catheter.

FIG. 14 is a cross-sectional schematic view of a thrombus retriever deployed on the distal side of a target thrombus within a blood vessel.

### DETAILED DESCRIPTION

The invention generally provides a thrombus retriever that is collapsible within, and is delivered by, an intravascular catheter. The retriever generally has a proximal end and a distal end. In some embodiments, a retriever body is connected to a deployment member (e.g., a central wire or other longitudinal backbone) that is under operator control and is adapted to translocate the retriever body longitudinally within the blood vessel lumen in order to collect the target thrombus. The retriever body is affixed to the deployment member such that pulling the deployment member in the proximal direction effectively “pushes” the retriever from behind (i.e., the distal end) in that proximal direction while generating a bias force outwardly that tends to push the retriever closer to the vessel wall for optimal collecting of dense thrombus attached to the vessel wall. In some embodiments, the retriever body is affixed at its distal end to the deployment member in order to generate the pushing force. In some embodiments, the retriever body also is connected to a collecting member (e.g., an inner tube) that is under operator control and is adapted to cause the retriever body to reenter the catheter with the collected thrombus and resume a crimped conformation once thrombus collection is complete. Generally, the collecting member is pulled in a proximal direction, applying tension to collecting wires attached to the retriever body. The collecting wires are configured to cause a reduction in the retriever body diameter to facilitate reentry of the retriever body and collected thrombus into the catheter lumen. In other embodiments, the retriever body and the collecting wires are attached to a common longitudinal backbone that facilitates both longitudinal translocation of the retriever within the vessel lumen and recovery of the retriever body within the catheter lumen after thrombus collection is complete. Various embodiments of the thrombus retriever are described in more detail below. The description of common elements in the context of one embodiment are equally applicable to other embodiments having those same or similar elements.

The three major improvements of the thrombus retrievers described herein are: (a) the retriever body is pushed from behind by the arms generating bias towards the vessel wall as opposed to a bias away from the wall in state of the art thrombus retrievers that are pulled from the front; (b) the cross-section of the frame is canted such that additional bias is created towards the vessel wall; and (c) the collecting net at the distal end of the retriever body for collecting the thrombus. Improvement (a) and (b) improve the efficiency of collecting hard thrombus next to the vessel wall and improvement (c) improves the collection of a soft thrombus and thrombus fractures to prevent emboli.

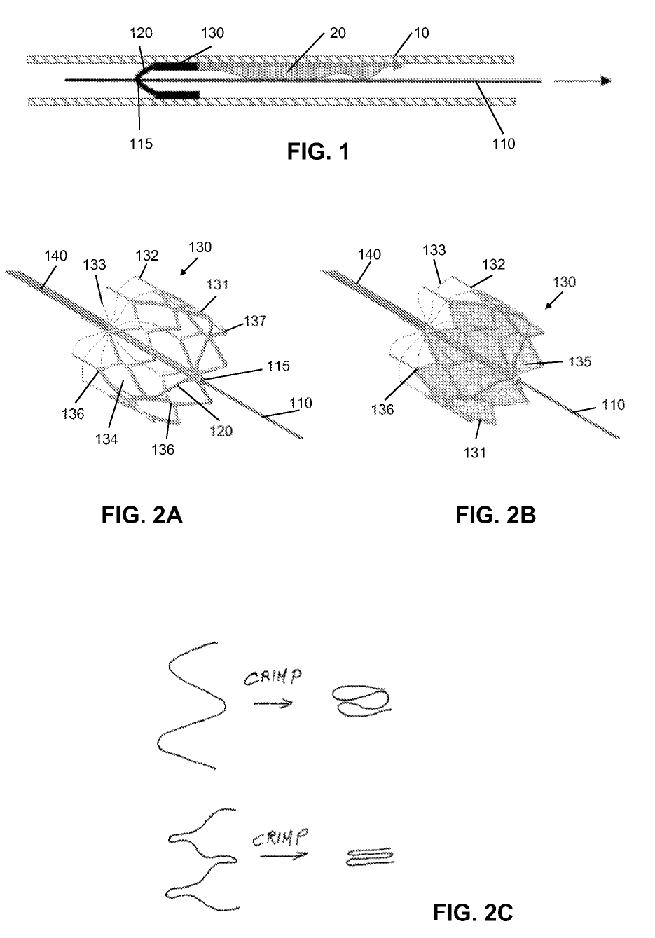

FIG. 1 is a cross-sectional schematic diagram of one embodiment of a thrombus retriever 100 constructed in accordance with some principles of this invention. The retriever 100 is illustrated in its deployed position on the distal side of the thrombus 20 within a blood vessel 10. The retriever 100 is formed from a deployment member, illustrated as central wire 110, a retriever body 130, and a plurality of distal arms 120. The distal arms 120 are attached at one end to the central wire 110 at an attachment point 115 that is distal to the retriever body 130, and at the other end to the retriever body 130. The retriever body 130 is generally cylindrical in shape and configured to circumnavigate the inner surface of the vessel 10. As described in more detail below, the retriever body 130 is partially or completely covered on its lateral and/or distal sides with a mesh adapted to trap the thrombus and thrombus debris once dislodged. When the retriever is pulled through thrombus 20 in the direction of the arrow, the arms 120 generate a bias towards vessel wall 10 and result in a more complete thrombus collection near the vessel wall.

In use, the operator pulls the central wire 110 in the proximal direction (arrow), causing the retriever body 130 to be translocated in the proximal direction. The pulling force applied by the operator is transferred from the central wire 110 to the retriever body 130 via the distal arms 120. The transfer of force causes the distal arms 120 to apply a slight outward (radial/lateral) pressure to the retriever body 130 which tends to maintain the retriever body pushed against the inner wall of the vessel 10. The retriever body 130 therefore tends to scrape the hard and dense portions of the thrombus perimeter from the inner wall of the vessel 10 as it is moved in the proximal direction. The retriever 100 and the thrombus and debris retained therein are collected into the catheter and removed from the body. Additional features of retriever 100 are described in more detail below.

FIG. 2 provides schematics of one embodiment of a thrombus retriever apparatus 100 that may be delivered, deployed, and retrieved using an intravascular catheter 30 (not illustrated). The thrombus retriever apparatus 100 consists of a retriever body 130, illustrated in a deployed conformation, an inner tube 140 which serves as a collecting member, and a deployment member 111 (e.g., a central wire 110), disposed through the lumen of the inner tube 140. The inner tube 140 and deployment member 111, at their proximal ends, are under independent and translational control by the operator in the longitudinal direction (i.e., may be translated in a direction substantially parallel to the central axis of the catheter 30 and vessel lumens). Independent control of these elements facilitates the deployment and retrieval of the apparatus 100.

FIG. 2A illustrates one embodiment in which the retriever body 130 is a self-expanding frame formed from struts 131 and a plurality of leading arms 132. The retriever body 130 and, optionally, the struts 131 are formed from a shape memory alloy or polymer such that the retriever body 130 can adopt a collapsed/crimped conformation and an expanded/deployed conformation. As discussed above, the retriever body 130 is substantially cylindrical and sized to circumnavigate the inner wall of the blood vessel of interest. The struts 131 are illustrated as forming a pattern of diamond- or parallelogram-shaped fenestrations 134 in which the struts 131 form a zigzag or substantially sinusoidal rings. In one embodiment, the retriever body is made of one or more (e.g., one, two, three, four, or more) first circumferential bands in which the struts form a first generally sinusoidal ring and one or more (e.g., one, two, three, four, or more) second circumferential ring. The first and second sinusoidal rings may or may not be identical. In one embodiment, the periodicity of the first and second sinusoidal patterns is the same. In another embodiment, the first and second circumferential bands are alternated over the length of the retriever body 130. Optionally, the first and second circumferential bands are aligned either in phase (loops of fenestrations 134 point in the same direction) or out of phase (loops pointing outside in opposing directions) and connected in a longitudinal direction to form a substantially cylindrical retriever body 130.

Optionally, the arms of the sinusoidal ring between loops is sigmoidal rather than straight in order to allow a smaller and denser crimped diameter. FIG. 2C illustrates suitable sinusoidal strut patters in their expanded and crimped conformations. In one embodiment the circumferential band is formed from a single sinusoidal strut in its expanded configuration. The circumferential band in its crimped conformation adopts a bulb-and-neck appearance (FIG. 2C, top). In another embodiment, each phase of the sinusoid is formed from tri-partite strut arranged approximately parallel to the longitudinal axis of the retriever body. In this embodiment, the terminal ends of each strut are substantially parallel to the longitudinal axis of the retriever body but offset from each other by a distance in the circumferential direction. The terminal ends of the struts are connected by an intermediate strut running at an angle. The result is a zig-zag strut pattern around the circumference of the retriever body. In its crimped conformation, each of the tripartite struts is aligned substantially parallel to the longitudinal axis of the retriever body. This conformation is particularly space-efficient and useful for thrombus retriever applications requiring very small crimped conformations (e.g., 1-2 mm in diameter) for use within small diameter blood vessels such as those found in the cranium and in and around the brain.

The retriever body 130 may be constructed of known materials, and for example stainless steel or cobalt chromium, but it is particularly suitable to be constructed from shape memory alloys such as NiTi. The pattern can be formed by laser cutting or etching a tube or flat sheet of material into the pattern shown. A flat sheet may be formed into a retriever body 130 by rolling the etched or laser cut sheet into a tubular shape, and welding the edges of the sheet together to form the tubular retriever body 130. The details of this method of forming the retriever body 130 are substantially the same as may be used to form intravascular stents and are disclosed in U.S. Pat. Nos. 5,836,964 and 5,997,703, each of which is hereby incorporated by reference in its entirety. Other methods known to those of skill in the art such as laser cutting a tube or etching a tube may also be used to construct a retriever body 130 in the present invention. When NiTi or certain other memory shape alloys are used, the retriever body 130 is heat treated after formation into a tubular shape, as known by those skilled in the art, to take advantage of the shape memory characteristics and/or super elasticity.

The foregoing pattern of struts 131 and fenestrations 134 is not intended to be limiting. Any suitable pattern of struts 131 and fenestrations 134 may be used. For example, wire frame retriever body 130 may have any pattern used to construct intravascular stents including, for example, the patterns disclosed in U.S. Pat. Nos. 6,197,048, 6,355,059, and 7,033,386, and U.S. Patent Publication 2012/0283817, each of which is hereby incorporated by reference. Furthermore, the retriever body 130 is illustrates has having two rows of struts 131 forming one row of fenestrations 134. This design choice is not limiting. A retriever body 130 may be constructed by increasing the number of rows of struts 131 and fenestrations 134, thereby forming a longer cylinder. The retriever body 130 cylinder formed by the struts 131 is open on its proximal and distal ends, notwithstanding the mesh covering 135 described in more detail below.

In one specific embodiment, the retriever body 130 is longitudinally translocated within the vessel lumen and deployed/pulled for thrombus retrieval using two separate elements, each under independent control of the operator. The first element is a deployment member (e.g., a longitudinal backbone), exemplified in this embodiment as deployment member 111. The deployment member generally serves to longitudinally translocate the retriever body 130 within the vessel lumen and/or catheter lumen, either in the crimped or deployed conformation. The retriever body 130 is rigidly attached to the deployment member and pulling the deployment member by the arms 120 in the proximal direction provides the motive “pushing” force on the distal side of the retriever body 130. Thus, the deployment member 111 is sufficiently rigid to accommodate that application of force. It is understood that, although exemplified as deployment member 111, the deployment member 111 need not be a tube and instead may be any suitable structure or shape such as a wire.

The second retriever body 130 control element is the collection member which is adapted to cause the retriever body to return to a crimped conformation from the deployed conformation once thrombus collection is complete. The collection member is under independent operator control and is functionally connected to the retriever body 130 by flexible wires 133 and collecting arms 132. The collection member is illustrated below as tube 140 and having the deployment member 111 disposed through its lumen. However, this configuration is not intended to be limiting. For example, the collection member may be a second rigid wire and wherein both the deployment member and the deployment member are independently disposed within the catheter lumen.

In the embodiment illustrated in FIG. 2, the deployment member, illustrated as deployment member 111, is longitudinally disposed through the lumen of the inner tube 140 and the central axis of the cylindrical retriever body 130. The retriever body 130 is attached at its distal end to an attachment point 115 on deployment member 111 via a plurality (e.g., two, three, four, five, six, or more) of distal arms 120. The attachment point 115 is distal to the distal end of the retriever body 130. The distal arms 120 extend from the attachment point 115, preferably in a symmetrical and/or radial pattern, in a proximal direction to distal edge of the retriever body 130. The angle formed by the distal arms 120 and deployment member 111 is preferably about 30°-70°. In one embodiment, the distal arms 120 are connected to the distal peaks 137 of the retriever body. Optionally, one distal arm 120 connects every distal peak 137 to deployment member 111 at the attachment point 115. Alternatively, one distal arm 120 connects every other distal peak 137 to deployment member 111 at the attachment point 115. The distal arms 120 may be constructed of the same or different material as the retriever body 130, and may be integral to the retriever body 130 during fabrication or individually attached as separate elements.

The distal arms 120 may be attached to the translocating member 111 in any appropriate manner. In some embodiments, the plurality of distal arms are attached to the same attachment point 115 on the deployment member 110 or to different (a plurality of) attachment points 115. For example, for embodiments in which at least two of the plurality of distal arms 120 have different lengths, a shorter distal arm 120 is attached to the deployment member at a first attachment point 115 that is closer to the retriever body 130, but still distal relative to the distal edge of the retriever body 130, and a longer distal arm is attached to the deployment member at a second attachment point 115 that is more distal on the deployment member 110 than the first attachment point 115.

Distal arms 120 may be permanently attached to the deployment member 110 by any suitable means. For example, the distal arms 120 may be fabricated as separate elements and welded at the attachment point and onto the retriever body 130. Alternatively, distal arms 120 may be fabricated as a contiguous component of either the deployment member 110 or the retriever body 130, and connected to the other component.

Leading arms 132 are rigid or semi-rigid struts attached at their distal ends to the proximal edge of the retriever body 130 and project in a direction that is substantially parallel to the central axis of the cylindrical retriever body 130. The leading arms 132 may be constructed of the same or different material as the retriever body 130, and may be integral to the retriever body 130 during fabrication or individually attached as separate elements. In one embodiment, the leading arms 132 extend from the proximal peaks 136 of the retriever body 130. In one embodiment, each proximal peak 136 has an attached leading arm 132. Optionally, leading arm 132 are adapted to have a connection point for wires 133 such as an eyelet or any other form.

Wires 133 are adapted to transition the retriever body 130 from the deployed to the crimped or collected conformations. Wires 133 preferably are arranged in a 1:1 relationship with leading arms 132, but other conformations are possible (e.g., two wires 133 are attached to each leading arm 132). Wires 133 are attached at their distal end to the proximal end of the leading arms 132 and at their proximal end to the inner tube 140. Attachment of wires 133 to inner tube 140 may be on the exterior surface, interior lumen, or distal edge of the inner tube 140. The wires 133 may be formed of any suitable material that is both flexible and capable of sustaining the force necessary to close the retriever body 130 from the deployed conformation to at least a partially-crimped conformation for collection. The wires 133 may be formed from a metal (e.g., NiTi) or a thermoplastic polymer, for example.

The inner tube 140 is sized to fit within the catheter lumen 35. In one embodiment, the outer diameter of inner tube 140 is slidingly fit into the inner diameter of the catheter lumen 35, thereby maximizing the cross-sectional area of the inner tube 140. The inner tube 140 may be made of any suitable material including, for example, metal or thermoplastic polymers or combination of polymer and metal braiding. Alternatively, the outer diameter of inner tube 140 is small enough to fit within the lumen of the retriever body 130 in the crimped conformation.

FIG. 2B illustrates a mesh covering 135 attached to the retriever body 130. The mesh covering 135 should cover at least the distal end of the retriever body 130. Optionally, the mesh covering 135 covers some or all of the fenestrations 134. As illustrated, the mesh covering 135 covers every fenestration 134 and the distal end of the retriever body 130. The mesh covering 135 is adapted to retain the collected thrombus 20 and associated debris within the cavity of the retriever body 130 as the thrombus 20 is dislodged from the vessel 10 wall. The mesh covering 135 may be a mesh of fine fibers (e.g. applied by electro-spinning) or a porous continuous coating or a woven material (e.g. Dacron) with adequate pore size. A mesh covering 135 of fine fibers may be constructed of metal or polymer (e.g., a thermoplastic polymer such as EPTFE, polyurethane, polyethylene, and nylon) fibers. In one embodiment, mesh covering 135 is constructed of wires of the same material as the retriever body 130 (e.g., a shape memory alloy such as nitinol). Porous continuous coatings include, for example, Dacron weaved net or continuous polymer membrane of different polymeric material (nylon, polyurethane, polyethylene, etc.).

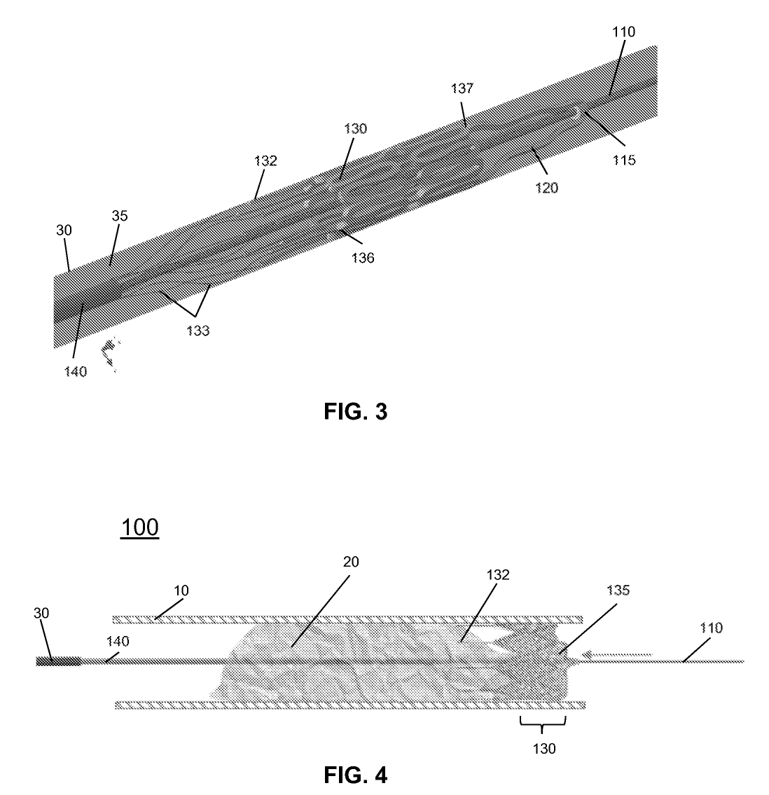

FIG. 3 illustrates a perspective view of the thrombus retriever 100, in its crimped conformation within a catheter lumen 35. For clarity, the mesh covering 135 is not shown. In this figure the deployment member is the central wire 110 itself. The retriever body 130 is illustrated as being formed from cylindrical struts fashioned into a generally sinusoidal ring and the struts may have a sigmoidal shape to decrease the diameter of the crimped retriever body. In this embodiment, wires 133 are untensioned and loose. Accordingly, the retriever body 130 will automatically expand to the deployed conformation as it exits the catheter lumen 35.

The principles of the construction and features of the thrombus retriever 100 are further illustrated by describing its operation. The thrombus retriever 100 is first placed in its crimped conformation. The thrombus retriever 100 is then loaded into the lumen 30 of an intravascular catheter 30 while the inner tube 140 and central wire 110 (or element 111) are held in a fixed relationship to each other. Optionally and if necessary, the inner tube 140 is move in the distal direction to relieve tension on the wires 133. This will allow the retriever body 130 to expand and adopt the deployed conformation immediately and automatically upon its exit from the catheter lumen 35.

The loaded catheter 30 is inserted into the patient's body in the standard manner (e.g. through the radial artery) and guided to the site of the target thrombus 20. The catheter 30 is pushed through the thrombus 20 until the catheter lumen 35 is across the thrombus. The thrombus retriever 100 is deployed on the distal side of the thrombus 20. In one embodiment, the thrombus retriever 100 is deployed by pushing the deployment member (e.g., central wire 110 or deployment member 111) in the distal direction while the catheter 30 sheath is maintained in a substantially fixed position relative to the blood vessel 10. After deployment, the catheter 30 sheath then is withdrawn to the proximal side across the thrombus 20 location retrieving the thrombus. In another embodiment, the retriever 100 is deployed on the distal side of the thrombus by withdrawing the catheter 30 sheath to the proximal side of the thrombus while maintaining the retriever body 130 in a substantially fixed position relative to the blood vessel 10 (i.e., by sliding the catheter 30 in the proximal direction relative to the deployment member 110 or 111.

FIG. 4 illustrates the thrombus retrieval process. Once deployed on the distal side of the thrombus 20, the retriever 100 is pulled in the proximal direction (arrow) to collect the thrombus 20 into the mesh covering 135.

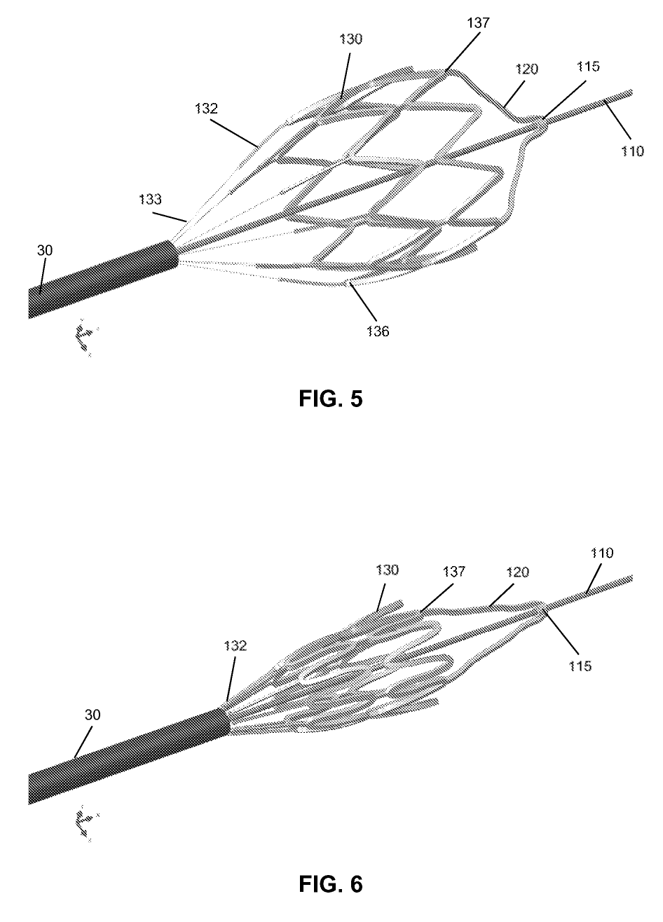

After the thrombus is retrieved (i.e., after the retriever body 130 has been translocated in the proximal direction to traverse all or a portion of the thrombus 20 to be retrieved), the inner tube 140 is pulled in the proximal direction to collect the retriever body and the thrombus into the catheter 35. FIG. 5 illustrates the retriever 100 in a partially collected conformation as it is being pulled into the lumen of the catheter 30. The inner tube 140 is withdrawn into the lumen 35 of the catheter 30 and is not visible in FIG. 5. Accordingly, wires 133 are tensioned causing an inward deflection of leading arms 132. Inner tube 140 and central wire 110 are further pulled to collect the retriever body 130 into the catheter 30. As illustrated in FIG. 6, the process is continued until the proximal ends of the leading arms 132 are enter the catheter lumen 35. At this point, the inner tube 140 and the central wire 110 can be pulled in the proximal direction in order to collect the retriever body 130, and the thrombus contained therein, fully within the catheter lumen 35. The catheter 30 is then removed from the body.

It is understood that the retriever body 130 may be returned only to a partially crimped conformation by the proximal pulling action of the inner tube 140 on the wires 133 during the retrieval process. To effect retrieval, it is sufficient for the pulling action of the inner tube 140 and wires 133 to translocate the leading arms 132 toward the central axis enough that the proximal ends of the leading arms 132 form a diameter smaller than the diameter of the catheter lumen 35, even if the retriever body 130 is not collapsed into its fully crimpled conformation. From this partially-crimped conformation, the proximal ends of the leading arms 132 may be pulled in the proximal direction, using the inner tube 140 and the central wire 110, so that the ends are disposed within the catheter lumen 35. Once so disposed, the remainder of the retriever apparatus 100 can be pulled in the proximal direction into the catheter lumen 35, completing the transition into the fully collected conformation.

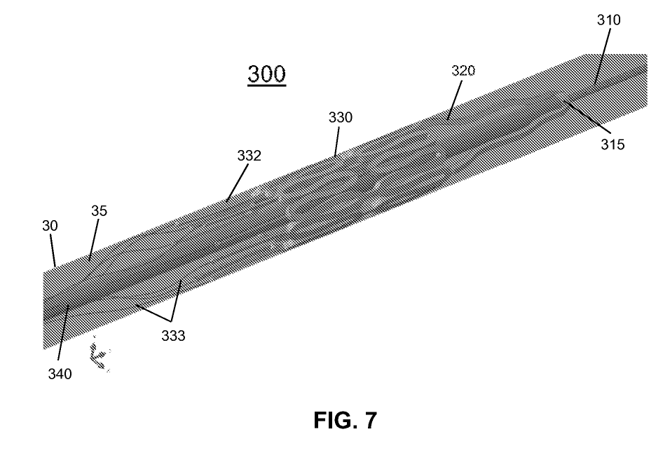

FIG. 7 illustrates a perspective view of another embodiment of the invention, thrombus retriever 300, in its crimped conformation contained within a catheter lumen 35. Retriever body 330, distal arms 320, leading arms 332, wires 333, and the mesh (omitted for clarity) are constructed and configured as described above. In this embodiment, distal arms 320 are attached to attachment point 315 on a centrally-disposed actuator 340, and wires 333 also are attached to actuator 340. As above, attachment point 315 is distal to the distal-most edge of retriever body 330. Actuator 340 may be a wire or a tube having a lumen. Optionally, in embodiments in which actuator 340 is a tube, guide wire 310 is disposed along the longitudinal axis of the actuator 340 lumen. Guide wire 310 may be used to guide the catheter and/or the retriever 300 through the blood vessel.

In use, catheter 30 is pushed through the thrombus 20 until the catheter lumen 35 is across the thrombus. The thrombus retriever 300 is deployed on the distal side of the thrombus 20. In one embodiment, the thrombus retriever 300 is deployed by pushing the actuator 340 in the distal direction while the catheter 30 sheath is maintained in a substantially fixed position relative to the blood vessel 10. After deployment, the catheter 30 sheath then is withdrawn to the proximal side of the thrombus 20. In another embodiment, the retriever 300 is deployed on the distal side of the thrombus by withdrawing the catheter 30 sheath to the proximal side of the thrombus while maintaining the retriever body 130 in a substantially fixed position relative to the blood vessel 10 (i.e., by sliding the catheter 30 in the proximal direction relative to actuator 340). In the deployed conformation, wires 333 should not be deflected by the distal edge 31 of catheter 30. Thrombus collection is achieved by pulling actuator 340 in the proximal direction to collect the thrombus 20 in the mesh covering.

After collection of the target thrombus, retriever body 330 is recovered into the catheter lumen 35 by a further translocation of actuator 340 in the proximal direction. Wires 333 become deflected by the distal edge 31 of catheter 30 as retriever body 330 nears the distal opening of catheter lumen 35. This proximally-directed translocation of retriever body 330 relative to catheter 30 causes wires 333 to inwardly defect leading arms 332, thereby reducing the diameter circumscribed by the proximal ends of leading arms 332. Continued proximal translocation of actuator 340, through the crimping action of wires 333 on leading arms 332, draws the proximal ends of leading arms 332 within the catheter lumen 35. Further proximal translocation of the actuator 340 then pulls the retriever body 330 fully within the lumen by the concomitant application of force through the attachment point 315 and distal arms 320, thereby collecting the retriever body 330 and collected thrombus within the catheter lumen 35.

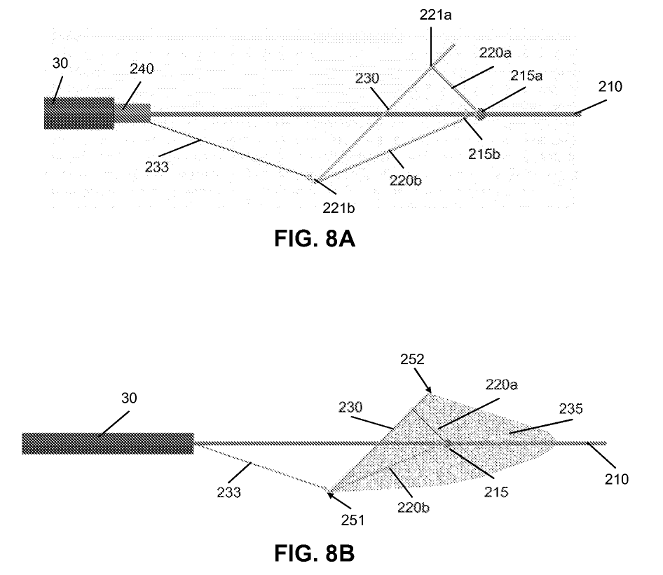

FIGS. 8-10 illustrate another embodiment of the invention in which the retriever body 230 is a generally circular or elliptical ring and configured to circumnavigate the inner surface of the vessel 10.

FIGS. 8A-8B illustrate a wire-style retriever body 230, wherein, in cross-section, retriever body 230 is substantially round and is approximately the same dimension in all planes. Specifically, FIG. 8A is a schematic diagram of a thrombus retriever 200 in a deployed conformation and constructed in accordance with the principles of this invention. The thrombus retriever 200 is illustrated in the deployed conformation extending from the catheter 30. The retriever apparatus 200 consists of a retriever body (ring) 230, an inner tube 240, and a central wire 210 or other suitable backbone disposed through the lumen of the inner tube 240. The inner tube 240 and central wire 210, at their proximal ends, are under independent and translational control by the operator in the longitudinal direction (i.e., may be translated in a direction substantially parallel to the central axis of the catheter 30 and vessel lumens). Independent control of these elements facilitates the deployment, retrieval and collection of the apparatus 200. The retriever body 230 is supported by a plurality of distal arms 220a, 220b, etc. extending from one or more attachment points 215 on the central wire 210 to various attachment points 221a, 221b, etc. on the retriever body 230. The attachment point(s) 215 is distal to the distal end of the retriever body 230. In some embodiments, the plurality of distal arms 220a, 220b, etc. are attached to the central wire 210 at a plurality of attachment points 215a, 215b and at least some of the attachment points 215 are slidably engaged with the central wire 210. The slidably engaged attachment point(s) 215 facilitates the use and fit of the deployed retriever body 230 into blood vessels of varying diameter by allowing for a variable angle of the retriever body 230 relative to the central wire 210. The retriever body 230 is attached to the inner tube 240 via a collecting wire 233. As illustrated in FIG. 8B, a net 235 is attached to the retriever body 230 and extends distally therefrom. For clarity, the net 235 is omitted from FIG. 8A.

Net 235 is attached to retriever body 230 and, in the deployed conformation, extends in the distal direction. Net 235 may have any suitable shape and dimension designed to accommodate the volume of the thrombus to be retrieved and that is capable of being collapsed into a crimped conformation and delivered/deployed from an intravascular catheter. The central wire 210 may pass through the net or on its side. The optional attachment of net 235 to central wire 210 is generally located in the distal portion of net 235 and preferably at its most distal point or face. Optionally, net 235 is attached to attachment point 215.

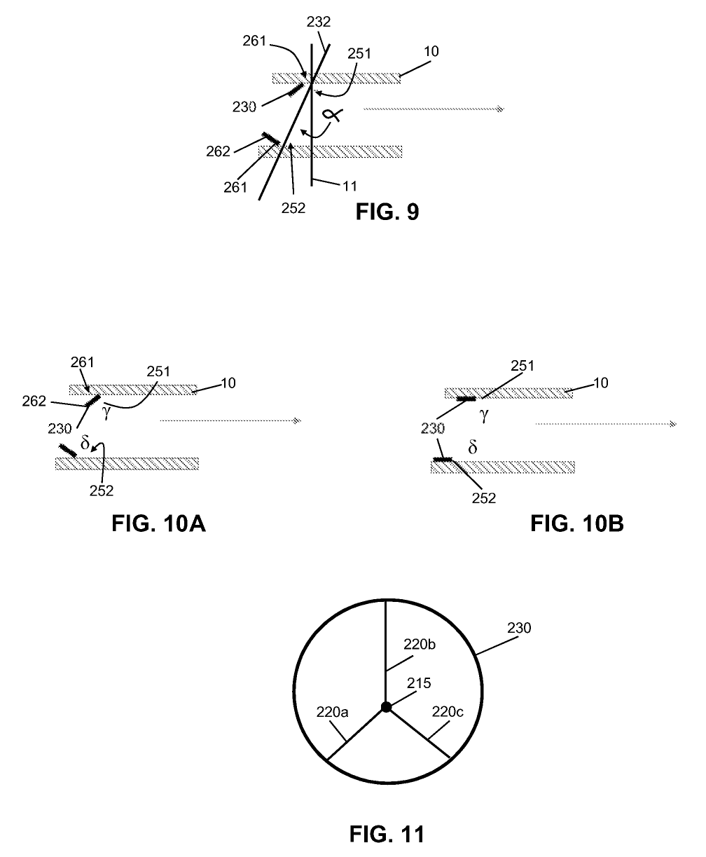

FIG. 9 is a cross-sectional diagram illustrating the orientation of the ribbon-style retriever ring 230 within the lumen of the blood vessel 10. The retriever body 230 contacts lumen wall of the vessel 10, preferably over its entire circumference. Retriever body 230 defines a proximal contact point 251 and a distal contact point 252 which are the most proximal and distal points, respectively, of contact between the proximal edge 261 of the retriever body 230 and the vessel wall 10. The retriever body plane 232 is defined by the plane of the ellipse and is determined by the ratio between the vessel diameter and the free diameter of the ring 230. The cross-sectional plane 11 is the plane orthogonal to the longitudinal axis of the vessel lumen, the longitudinal axis of the retriever 200, longitudinal axis of the retriever body 230 without regard to the cant described below, and/or the deployment member 210. The retriever body plane 232 may be canted to form an angle α which is about 0°-45° (e.g., about 5°, 10°, 15°, 20°, 25°, 30°, 35°, 40°, or 45°) relative to the cross-sectional axis 11.

FIGS. 9 and 10 illustrate a ribbon-style retriever body 230 in cross-section. The retriever body 230 is an ellipse slanted in the longitudinal direction of the vessel to fit the perimeter of the vessel. Although wire- and ribbon-shaped retriever bodies 230 are illustrated herein, the cross-sectional shape of the retriever body is not intended to be limiting. For example, suitable retriever bodies 230 may be square or even formed from a plurality of twisted or braided wires. Retriever body 230 is generally designed to provide a proximal edge 261 adapted to dislodge the thrombus 20 from the inner wall of the vessel 10 and further adapted to provide attachment points 221a, 221b, 221c, etc. for the distal arms 220a, 220b, 220c, etc. The distal arms 220 and attachment point(s) 221 may be configured as described for the embodiment illustrated in FIG. 8. Materials for the retriever body and the net, as well as their manufacture from one piece or connected parts are as described above for the previous embodiments.

FIG. 10 is another cross-sectional diagram illustrating the Advantage of the ribbon cross-section being pointed outwards (FIG. 10A) as opposed to being parallel to the vessel wall (FIG. 10B). The FIG. 10A configuration will tend to retrieve more completely the dense thrombus in a single sweep. FIG. 10 is another cross-sectional diagram illustrating the positioning of a ribbon-style retriever body 230 within the lumen of the blood vessel 10. The retriever body 230 is illustrated as a flat ribbon having a proximal edge 261 and a distal edge 262. As illustrated in FIG. 9, the retriever body 230 contacts lumen wall of the vessel 10, preferably over its entire circumference and that contact defines a proximal contact point 251 and a distal contact point 252. In this embodiment, the distal edge 262 of the retriever body 230 is medially-disposed related to the proximal edge 261 such that the retriever body forms cant angles γ and δ at the proximal contact point 251 and a distal contact point 252, respectively. The cant angles γ and δ may be the same or different and generally are between about 135°-180° (e.g., about 140°, 150°, 160°, or 170°) relative to the vessel wall extending in the proximal direction (arrow). It is understood that, when angles γ and δ are different, the contact angle continuously varies around the circumference of the retriever body 230 between the values of angles γ and δ. FIG. 10A illustrates one embodiment in which angles γ and δ are less than 180°. FIG. 10B illustrates an embodiment in which angles γ and δ are substantially equal to 180°. The maximum and minimum values for angles γ and δ, when different, need to be at contact points 251 and 252 as is illustrated herein for convenience. An angle substantially smaller than 180° is preferred as it will generate a bias towards the vessel wall as the ring is being pulled through the thrombus, but this should not be a limitation.

A plurality (e.g., three, four, five, six, or more) of distal arms 220 are attached at their proximal end to the distal edge of retriever body 230 and at their distal end to the central wire 210 at attachment point 215. Distal arms 220 may be attached to the same or different attachment points 215. For example, as illustrated in FIG. 8A, distal arm 220a is joined to attachment point 215a and distal arm 220b is joined to attachment point 215b. The location of the attachment points 215 on central wire 210 is not limited except that distal arms 220 must project in the proximal direction for attachment to retriever body 230 and the attachment of the respective arms 220 must be gliding and independent to allow the transition of 230 from the crimped conformation to the deployed one and also allow different deployed conformations for different vessel diameters. Distal arms 220 provide a “pushing force” in the proximal direction when the central wire is pulled in the proximal direction by the operator and help to retrieve more thrombus next to the vessel wall and generate an outward bias.

FIG. 11 is a top plan view of one embodiment of the retriever 200. In this embodiment, retriever body 230 is attached to the attachment point 215 on the central wire 210 by three distal arms 220. Angle α (FIG. 9) may be controlled when the retriever 200 is in the deployed position by the selection of the lengths of the distal arms 220 and their relative positioning. As illustrated in FIG. 11, distal arms 220a and 220c are substantially the same length and distal arm 220b is substantially longer. When deployed, this configuration of distal arms 220 results in an angle α (FIG. 9) that is greater than 0° and the section of the ring attached to 220b will be more proximal than the section between 220a and 220c. In this configuration 215a and 215c may be common or different attachment points but 215b preferably is different and is gliding independently as the retriever body 230 deploys.

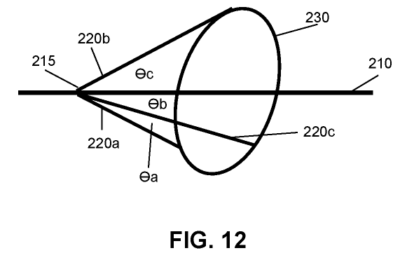

FIG. 12 is a schematic view of the retriever 200 in its deployed conformation. Distal arms 220a,b,c form angles Θa, Θb, and Θc, respectively. Angles Θ are independently determined and depend upon the distance in the distal direction of attachment point 215 from the retriever body axis 232 and/or the length of the individual distal arms 220. In some embodiments, angles Θ are about 30° to about 60°.

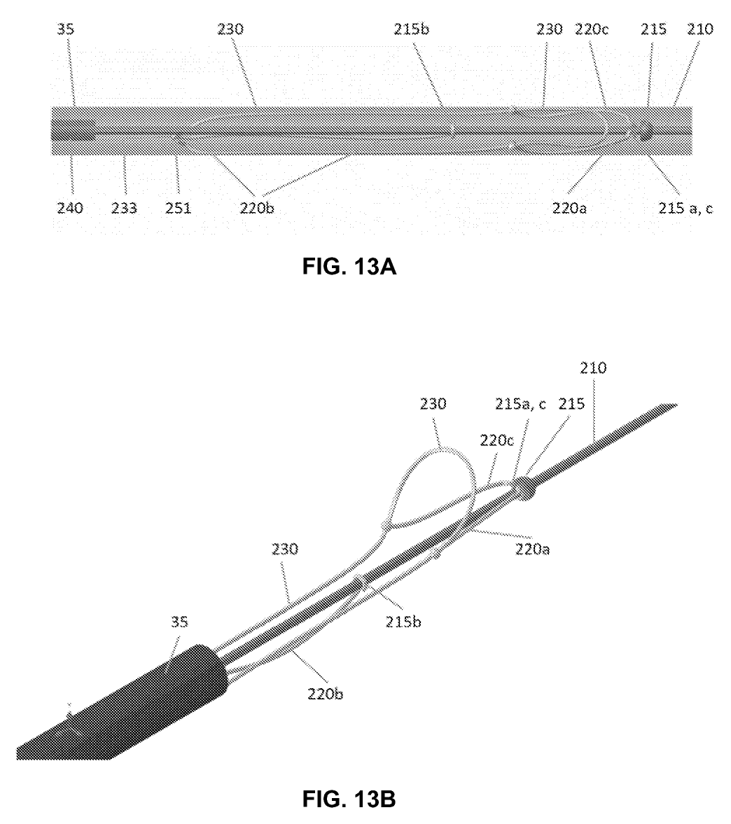

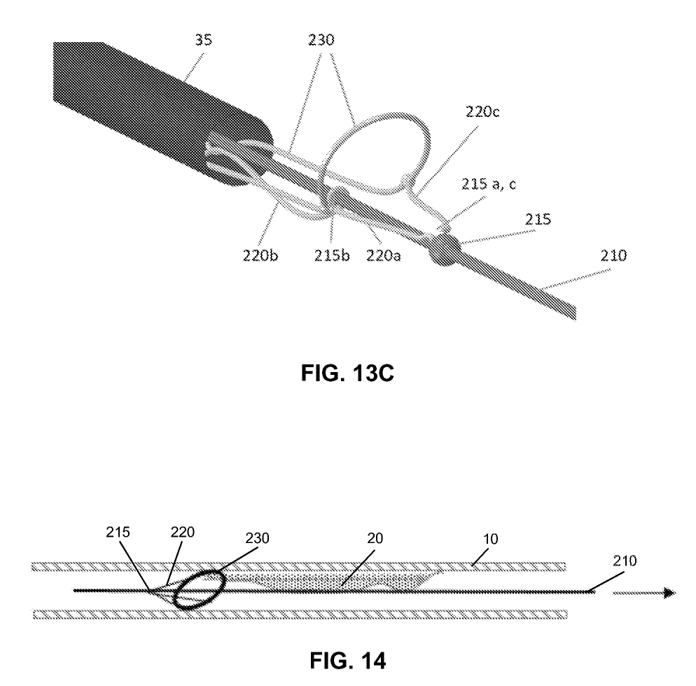

FIG. 13A illustrates the retriever 200 in a crimped conformation with the net 235 omitted for clarity. For deployment the retriever is pulled out of the catheter 35 (i.e., by the distal arms under the sliding action of central wire 210), the ring 230 assumes an elliptical shape as constrained by the vessel around it, with the attachment point 215b sliding to a position next to 215a and 215c at the attachment point 215. Once deployed the retriever can be pulled proximally to retrieve the thrombus with the distal arms 220 a,b,c pushing the ring 230 from behind and generating an outwardly force pushing the ring to the vessel wall for more complete retrieval of thrombus. When all thrombus has been retrieved into the net (not shown in FIG. 13) the retriever and the thrombus in it are collected into the catheter 35 by pulling the tube 240 and with it the collecting wire 233. The attachment points 215 will glide independently on the central wire 210 as the retriever body 230 transitions from crimped to deployed and back. The stages of collecting the retriever 200 into the catheter are shown in FIGS. 13B and 13C.

FIG. 14 schematically illustrates retriever 200 deployed on the distal side of the thrombus 20 within blood vessel 10. The retriever 200 generally functions in a manner similar to the previous embodiment. After deployment, the operator pulls the retriever 200 in the proximal direction (arrow) in order to dislodge the thrombus which is caught in the net 235 (omitted for clarity). The retriever 200 and the thrombus and debris retained therein are collected and removed from the body. When pulled proximally (arrow) the arms 220 are pushing the ring 230 outwardly to press against the vessel wall and have a more complete retrieval of thrombus attached to the wall.

For collection of retriever 200 into the catheter following thrombus collection, collecting wire 233 is tightened by translocating the inner tube 240 in the proximal direction relative to central wire 210 and catheter 35 until retriever body 230 is withdrawn into lumen 35 by translocating inner tube 240 (see, FIG. 13) and central wire 210 together in the proximal direction. FIGS. 13A-13C that show the deployment steps of this embodiment can also be seen as steps of collecting the retriever into the catheter, in the reverse order.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

What is claimed is:

1. A thrombus retriever comprising:
a substantially cylindrical retriever body open at a proximal end, open at a distal end, having a longitudinal axis, and capable of adopting a crimped conformation having a first diameter and a deployed conformation having a second diameter, and wherein the first diameter is smaller than the second diameter, and
a deployment member attached to the retriever body through a plurality of arms and configured such that a pulling force applied to the deployment member creates an outwardly-directed force on the retriever body.

2. The thrombus retriever of claim 1, further comprising a covering at the distal end of the retriever body.

3. The thrombus retriever of claim 2, wherein the covering is a mesh of polymer fibers, metal wires, or a membrane with perforations.

4. The thrombus retriever of claim 1, wherein the retriever body is self-expanding.

5. The thrombus retriever of claim 1, wherein the crimped conformation has a diameter of 1-6 French.

6. The thrombus retriever of claim 1, wherein the deployed conformation has a diameter of 3-10 French.

7. The thrombus retriever of claim 1, wherein the deployed conformation has a diameter of 10-20 French.

8. The thrombus retriever of claim 1, wherein deployment member extends through the retriever body along an axis substantially parallel to the longitudinal axis of the retriever body.

9. The thrombus retriever of claim 8, wherein the retriever body is attached to the deployment member by three or more arms, wherein the arms extend from the distal end of the retriever body to an attachment point on the deployment member that is distal to the distal edge of the retriever body.

10. The thrombus retriever of claim 1, wherein the retriever body comprises one or more circumferential bands having loops forming a generally sinusoidal or zig-zag pattern around the circumference of the retriever body, wherein consecutive circumferential bands maybe out of phase or in-phase.

11. The thrombus retriever of claim 1, wherein the retriever body further comprises a two or more leading arms extending from the proximal end of the retriever body and are connected to the deployment member.

12. The thrombus retriever of claim 11, wherein each of the leading arms is attached to the deployment member by one or more flexible wires.

13. The thrombus retriever of claim 1, wherein the retriever body further comprises a collection member attached to two or more leading arms extending from the proximal end of the retriever body and are connected to the collection member.

14. The thrombus retriever of claim 13, wherein each of the leading arms is attached to the collection member by one or more flexible wires.

15. A thrombus retriever comprising:
a substantially circular or elliptical retriever body capable of adopting a first crimped conformation and a deployed conformation, wherein the retriever body is a ring or a ribbon, and
a deployment member attached to the retriever body through a plurality of arms and configured such that a pulling force applied to the deployment member creates an outwardly-directed force on the retriever body.

16. The thrombus retriever of claim 15, further comprising a covering at the distal end of the retriever body.

17. The thrombus retriever of claim 16, wherein the covering is a mesh of polymer fibers, metal wires, or a membrane with perforations.

18. The thrombus retriever of claim 16, wherein the covering is bursiform.

19. The thrombus retriever of claim 15, wherein the retriever body is self-expanding.

20. The thrombus retriever of claim 15, wherein the crimped conformation has a diameter of 1-6 French.

21. The thrombus retriever of claim 15, wherein the deployed conformation has a diameter of 3-10 French.

22. The thrombus retriever of claim 15, wherein the deployed conformation has a diameter of 10-20 French.

23. The thrombus retriever of claim 15, wherein the retriever body is attached to the deployment member by two or more arms, wherein the arms extend from the distal end of the retriever body to an attachment point on the deployment member that is distal to the distal edge of the retriever body.

24. The thrombus retriever of claim 15, wherein the retriever body plane is canted at an angle of 0°-45° relative to the collecting member.

25. The thrombus retriever of claim 15, wherein the retriever body is a ribbon having a proximal edge and a distal edge, wherein the distal edge is medially-disposed relative to the proximal edge such that the ribbon forms a canting angle of 135°-180°.

26. The thrombus retriever of claim 15, wherein the collecting member is attached to the retriever body by one flexible wire.
